(19) United States
(12) Patent Application Publication
(10) Pub. No.: US 2025/0281380 A1
(43) Pub. Date: Sep. 11, 2025

(54) Skincare Formulation for Multi-Modal Reduction of Acetylcholine Concentration and Activity

(71) Applicant: JAMRM, LLC, Dallas, TX (US)

(72) Inventors: John Koch, Fort Worth, TX (US); Tatiana Kelly, Westlake, TX (US); Thu Q. Nguyen, North Richland Hills, TX (US); Alisar Salah Zahr, Dallas, TX (US)

(21) Appl. No.: 19/216,967

(22) Filed: May 23, 2025

Related U.S. Application Data
(63) Continuation of application No. 17/164,712, filed on Feb. 1, 2021, now abandoned.

(51) Int. Cl. A61K 8/64; A61K 8/06; A61K 8/44

(52) U.S. Cl. CPC A61K 8/64; A61K 8/064; A61K 8/066; A61K 8/44

(57) ABSTRACT

A skincare formulation for reducing facial expression lines via multi-modal reduction of ACh concentration and activity. The formulation includes a carrier base, an inhibitory neurotransmitter and a plurality of neuromodulating peptides configured to inhibit ACh concentration and activity at the epidermis as well as at the neuro-muscular junction. The inhibitory neurotransmitter is configured to reduce neuronal excitability. The plurality of neuromodulating peptides includes a first neuromodulating peptide configured to compete with SNAP-25 in the SNARE complex of the motor neuron at the presynaptic terminal, a second neuromodulating peptide configured to destabilize the SNARE complex at the presynaptic terminal, a third neuromodulating peptide configured to inhibit fusion of ACh vesicles in neuron plasma membrane, a fourth neuromodulating peptide configured to inhibit contraction of muscles via inhibition of transmitter-gated ion channel communication at the postsynaptic membrane, and a fifth neuromodulating peptide configured to block reception of ACh at the muscle cell, the postsynaptic membrane.

### STATEMENT OF RELATED APPLICATIONS

This application is a continuation of U.S. Ser. No. 17/164,712, filed on Feb. 1, 2021, of which is incorporated herein by reference in its entirety.

### BACKGROUND

### Technical Field

Novel aspects of the present disclosure relate to a formulation targeting facial expression lines for anti-aging purposes. More particularly, the present disclosure is directed to a formulation including for attenuating muscle contraction via a skincare formulation that provides a multi-modal reduction of acetylcholine concentration and activity.

### Background

Human skin is a multi-layered tissue that is complex in terms of its structure as well as in terms of its biochemistry. As skin ages over time, its visible appearance and texture changes. These changes are affected by the surrounding environment as well as the repeated stimulation of muscles, particularly in the face, resulting in the manifestation of fine lines and wrinkles. People have attempted to use cosmetic, topically applied formulations to minimize or reverse these appearance and texture changes. As the study of the human skin continues to progress, novel treatments for ameliorating skin aging are being developed.

### SUMMARY OF THE INVENTION

Novel aspects of the present invention are directed to a formulation for attenuating muscle contractions via multi-modal reduction of acetylcholine (ACh) concentration and activity. In one embodiment, the formulation includes a carrier base, an inhibitory neurotransmitter, and a plurality of neuromodulating peptides that inhibit ACh concentration and activity throughout the skin, particularly at the epidermis and the neuro-muscular junction. The inhibitory neurotransmitter is configured to reduce neuronal excitability. The plurality of neuromodulating peptides includes a first neuromodulating peptide configured to compete with SNAP-25 in the SNARE complex of the motor neuron (pre-synapse), a second neuromodulating peptide configured to destabilize the SNARE complex in the motor neuron (pre-synapse), a third neuromodulating peptide configured to inhibit fusion of ACh vesicles with the motor neuron plasma membrane (pre-synapse), a fourth neuromodulating peptide configured to inhibit contraction of muscles in an electrical-chemical communication of the muscle cell (post-synapse), and a fifth neuromodulating peptide configured to block reception of ACh at the muscle cell, the postsynaptic membrane (post-synapse).

Other aspects, embodiments, and features of the invention will become apparent from the following detailed description of the invention when considered in conjunction with the accompanying figures. In the figures, each identical or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE FIGURES

The novel features believed characteristic of the invention are set forth in the appended claims. The invention itself, however, as well as a preferred mode of use, further objectives, and advantages thereof, will be best understood by reference to the following detailed description of illustrative embodiments when read in conjunction with the accompanying figures, wherein:

FIG. 1 is a chart showing results of a test formulation for multi-modal reduction of ACh concentration and activity in accordance with an embodiment of the disclosure. The test utilizes a full thickness tissue model (MatTEK™ FT) which suggests in vivo inhibition of muscle contractions resulting in minimization of facial expression lines.

FIG. 2 is a chart comparing results of the test formulation for multi-modal reduction of ACh concentration and activity according to an embodiment of this disclosure with results derived from a prior art formulation providing only two mechanisms of action.

### DETAILED DESCRIPTION

Acetylcholine (ACh) is an organic chemical that functions as an excitatory neurotransmitter in the human body. ACh can be found in small, circular vesicles within the axon terminal of motor neurons, i.e., the nerve terminal at the pre-synaptic cell. A SNARE complex, also found in the motor neuron of the nerve terminal, is responsible for mediating the exocytosis and release of ACh into the neuro-muscular junction. The SNARE complex is formed from three proteins: vesicle associated membrane protein (VAMP), Syntaxin, and synaptosomal-associated protein, 25 kDa (SNAP-25). Release of ACh into the neuro-muscular junction between the nerve terminal of the presynaptic cell and the muscle cell causes muscle contraction when a threshold amount of ACh binds to ACh receptors at the post-synaptic membrane of the muscle cell to cause a conformational change in a membrane channel that is specifically permeable to both Na+ and K+. The increased permeability of these ions causes a depolarization of the post-synaptic membrane, which can initiate an action potential that causes muscle contraction.

Over time, repeated muscle contraction can be manifested as wrinkles and fine lines in the skin, particularly in the face. Reducing the frequency or severity of facial muscle contractions or attenuating the severity of the contractions can reduce the expression of wrinkles and fine lines. To this end, previous anti-aging skincare formulations included compounds that only inhibited the release of ACh from the presynaptic terminal into the neuromuscular junction and inhibited the binding of ACh at the post-synaptic membrane. However, these formulations failed to address another source of ACh production that can affect muscle contraction—keratinocytes.

Keratinocytes, the primary cell type found in the epidermis, synthesize, secrete, and degrade ACh that can be received into the neuromuscular junction. Presence of ACh in the neuromuscular junction from keratinocytes can increase the severity and frequency of muscle contractions, promoting the early onset of fine lines and wrinkles. Accordingly, novel aspects of the illustrative embodiments disclosed herein recognize a need for skincare formulations including compounds that also reduce the production of ACh from human keratinocytes.

Additionally, previous anti-aging skincare formulations operated under the assumption that the primary mechanism of action was the competition for binding sites for various proteins involved in the SNARE complex formation as well as destabilizing the SNARE complex in the release of ACh from the nerve terminal of the presynaptic cell and the binding of ACh at the post-synaptic membrane. Thus, the previous formulations included a fewer number of neuromodulating peptides, resulting in a narrower approach with fewer mechanisms of action targeting only the motor neuron and muscle cell. This narrower approach would be less effective in reducing concentration of ACh and thus minimizing the inhibition of muscle contractions. Accordingly, novel aspects of the illustrative embodiments disclosed herein also recognize a need for skincare formulations that could leverage the reduction of ACh from keratinocytes to maximize the reduction in ACh concentration and activity, thus minimizing the potential for muscle contraction and facial expression line formation.

### Exemplary Formulation

An embodiment of the formulation described herein includes active ingredients that work in concert to achieve a multi-modal reduction in ACh concentration and activity to attenuate muscle contraction, which reduces facial expression lines. As used herein, the reduction of ACh concentration and activity includes the inhibition of ACh release from sources such as keratinocytes and nerve terminals of the presynaptic cell, as well as the inhibition of ACh binding with ACh receptors at the post-synaptic membrane of muscle cells. More particularly, the active ingredients focus on three locations of action, namely the keratinocytes, the motor neurons, and the muscle cells, by inhibiting the release of ACh at the epidermal layer, throughout the skin including the dermal-epidermal junction and dermis, interfering with the SNARE complex at the nerve terminal of the presynaptic cell, inhibiting ACh release into the neuromuscular junction from the nerve terminal of the presynaptic cell, and destabilizing the ACh receptor at the post-synaptic membrane to inhibit ion transfer. The reduction of ACh in the neuromuscular junction originating from keratinocytes is a novel mechanism of action that can be paired with mechanisms of action targeting the motor neuron and muscle cell. Thus, exemplary formulations described herein can provide for an additional mechanism of action targeting another location of action as compared to previous formulations.

In one embodiment, the formulation includes active ingredients dispersed in a carrier, which can be a water-based or an anhydrous oil-based gel or an oil-in-water, water-in-oil, water-in-silicone, or water-free emulsion. The carrier base can form about 65 w/w % to about 99.6 w/w % of the formulation.

The active ingredients can include inhibitory neurotransmitters configured to reduce neuronal excitability and a plurality of neuromodulating peptides that provide three separate mechanisms of action targeting different locations. It is hypothesized that, when combined, the neuromodulating peptides and inhibitory neurotransmitter reduce ACh concentration and activity by binding with receptors (Nicotinic and Muscarinic) at the epidermal surface and/or are activating acetylcholinesterase, an enzyme which degrades acetylcholine. This leads to a more effective reduction of ACh concentration and activity at the epidermis and throughout the skin than could be achieved with a formulation lacking these compounds.

The inhibitory neurotransmitters bind to neurotransmitter receptors in the epidermal layer, which results in relaxation of expression muscles. In a non-limiting embodiment, the inhibitory neurotransmitter can be gamma aminobutyric acid (GABA) that binds with GABA receptors on the epidermal layer. When a skincare formulation includes GABA as the inhibitory neurotransmitter, GABA can be included in an amount between 0.1 w/w %-1 w/w % of the formulation.

The neuromodulating peptides configured to destabilize the SNARE complex reduce ACh activity by binding with the SNARE complex in place of SNAP-25, resulting in destabilization that inhibits ACh release into the neuromuscular junction. In a non-limiting embodiment, the neuromodulating peptides that compete with SNAP-25 in the SNARE complex include acetyl hexapeptide-8, which mimics the N-terminal end of SNAP-25 to compete with the SNAP-25 binding site in the SNARE complex. When a skincare formulation includes acetyl hexapeptide-8 as a neuromodulating peptide for destabilizing SNARE complex, acetyl hexapeptide-8 can be included in an amount between 0.001 w/w %-0.01 w/w % of the formulation.

In a non-limiting embodiment, the neuromodulating peptides that reduce ACh activity by competing with SNAP-25 in the SNARE complex include acetyl octapeptide-3, which also mimics the N-terminal end of SNAP-25. Acetyl octapeptide-3 is an elongation of acetyl hexapeptide-8. When a skincare formulation includes acetyl octapeptide-3 as another neuromodulating peptide for destabilizing SNARE complexes, acetyl octapeptide-3 can be included in an amount between 0.001 w/w %-0.01 w/w % of the formulation.

The neuromodulating peptides configured to inhibit neuronal transmission at the post-synaptic terminal reduce ACh activity by blocking the ACh receptors at the muscle surface which are responsible for controlling diffusion of ions that initiate muscle contraction. In a non-limiting embodiment, the neuromodulating peptides configured to inhibit neuronal transmission at the post-synaptic terminal include dipeptide diaminobutyroyl benzylamide diacetate (DDBD). DDBD mimics the essential amino acid sequence of the Waglerin-1 functionality and blocks neuronal transmission on the nicotinic ACh receptor, which prevents the ion channels in the post-synaptic membrane from opening. As a result, diffusion of sodium ions is blocked, which attenuates muscle contraction. When a skincare formulation includes DDBD as a neuromodulating peptide for inhibiting neuronal transmission at the post-synaptic terminal, DDBD can be included in an amount between 0.005 w/w %-0.01 w/w % of the formulation.

The neuromodulating peptides configured to inhibit fusion of ACh vesicles with the presynaptic membrane reduce ACh activity by inhibiting the release of ACh from the nerve terminal of the presynaptic cell into the neuromuscular junction. In a non-limiting embodiment, the neuromodulating peptides configured to inhibit fusion of ACh vesicles with the presynaptic membrane include palmitoyl hexapeptide-19, which inactivates SNAP-25 from the SNARE complex. Inactivation of the SNAP-25 inhibits fusion of ACh-filled vesicles with the plasma membrane at the neuromuscular junction, which in turn inhibits release of ACh. When a skincare formulation includes palmitoyl hexapeptide-19 as a neuromodulating peptide inhibiting fusion of ACh vesicles with the presynaptic membrane, palmitoyl hexapeptide-19 can be included in an amount between 0.0001 w/w %-0.0005 w/w % of the formulation.

The neuromodulating peptides configured to block reception of ACh at a postsynaptic membrane (muscle cell) reduce ACh activity by reducing ACh receptor clustering and destabilizing the ACh receptor, which inhibits calcium ion (CA+) release and attenuates muscle contraction via reduction of myosin fibers. In a non-limiting embodiment, when the neuromodulating peptide configured to block reception of ACh at a postsynaptic membrane is acetyl hexapeptide-1, the neuromodulating peptide can also reduce ACh activity at the nerve terminal of the presynaptic cell by binding with Syntaxin, which interferes with the ability for Syntaxin to bind to mammalian uncoordinate-18 (MUNC-18) in the SNARE complex, which inhibits SNARE complex formation. When a skincare formulation includes acetyl hexapeptide-1 as a neuromodulating peptide configured to block reception of ACh at a postsynaptic membrane, acetyl hexapeptide-1 can be included in an amount between 0.001 w/w %-0.005 w/w % of the formulation.

### Testing

The MatTek FT™ tissue model was used to determine whether treatment with the test formulation would impact ACh release in vitro. The MatTek FT™ tissue model is a three-dimensional, full-thickness model of human skin formed from cultured human epidermal keratinocytes and human dermal fibroblasts. The epidermal and dermal layers are mitotically and metabolically active and exhibit in vivo-like morphological and growth characteristics, which are uniform and highly reproducible. The MatTek FT™ tissue model allows for the performance of preclinical studies to evaluate mechanisms of action for skincare products. The test formulation that was used is shown in Table 1.

TABLE 1
Test Formulation.
TEST FORMULATION

| COMPOUND | AMOUNT (w/w %) |
|---|---|
| Purified Water | 96.7 |
| Glycerin | 2.0 |
| Gamma Aminobutyric Acid | 0.4 |
| Acetyl Hexapeptide-8 | 0.005 |
| Acetyl Octapeptide-3 | 0.003 |
| Dipeptide Diaminobutyroyl Benzylamide | 0.008 |
| Diacetate | |
| Palmitoyl Hexapeptide-19 | 0.0003 |
| Acetyl Hexapeptide-1 | 0.003 |

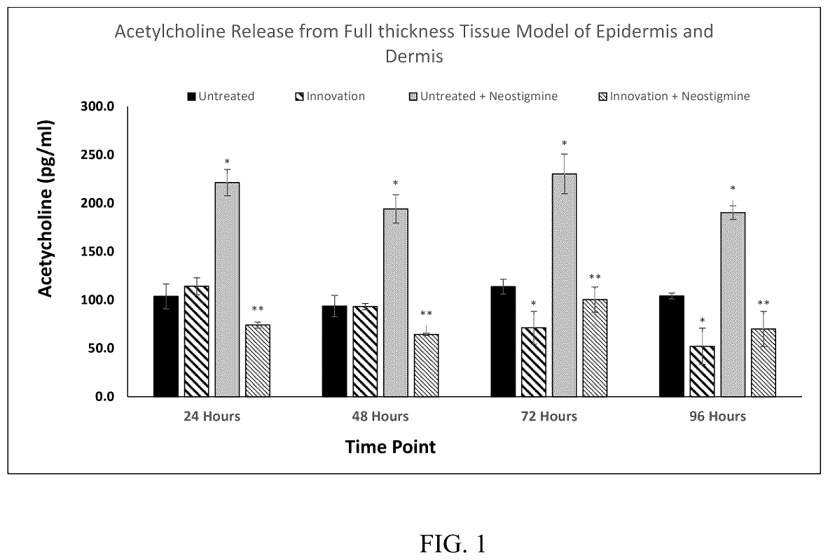

A first untreated tissue model served as a control sample and was maintained in a tissue culture media. A second tissue model was maintained in a tissue culture media and treated daily with the test formulation. A third tissue model was maintained in a tissue culture media and exposed to Neostigmine, an acetylcholinesterase inhibitor that prevents the breakdown of ACh in the tissue culture media. A fourth tissue model was maintained in a tissue culture media, treated daily with the test formulation, and exposed to Neostigmine. The amount of ACh created by each of the tissue models was determined by measuring the ACh concentration in the tissue culture media. The results can be seen in FIG. 1.

FIG. 1 is a chart showing results of a test formulation for multi-modal reduction of ACh activity for attenuating muscle contractions in accordance with an embodiment of the disclosure. The chart compares the concentration of ACh in picograms per milliliter versus time in 24 hour increments. The first untreated tissue model produced accumulated approximately 100 picograms of ACh per ml of solution. When compared to first untreated tissue model, the second tissue model showed that the accumulation of ACh was significantly reduced by the application of the test formulation by the third day, to about 75 picograms per ml of solution, and the accumulation of ACh remained reduced on the fourth day, at about 50 picograms per ml of solution. The third tissue model showed significant accumulation of ACh in the presence of the acetylcholinesterase inhibitor, to about 200 picograms per ml of solution, which was to be expected in the absence of ACh breakdown. The fourth tissue model showed that the accumulation of ACh was significantly reduced, to a concentration between 75-100 picograms per ml of solution, as compared to the third tissue model. The resultant conclusion is that the test formulation is capable of reducing the release of ACh from keratinocytes, in which five neuromodulating peptides and an inhibitory neurotransmitter are optimally formulated to be incorporated into skincare formulation for attenuating muscle contraction via multi-modal reduction of ACh concentration and activity.

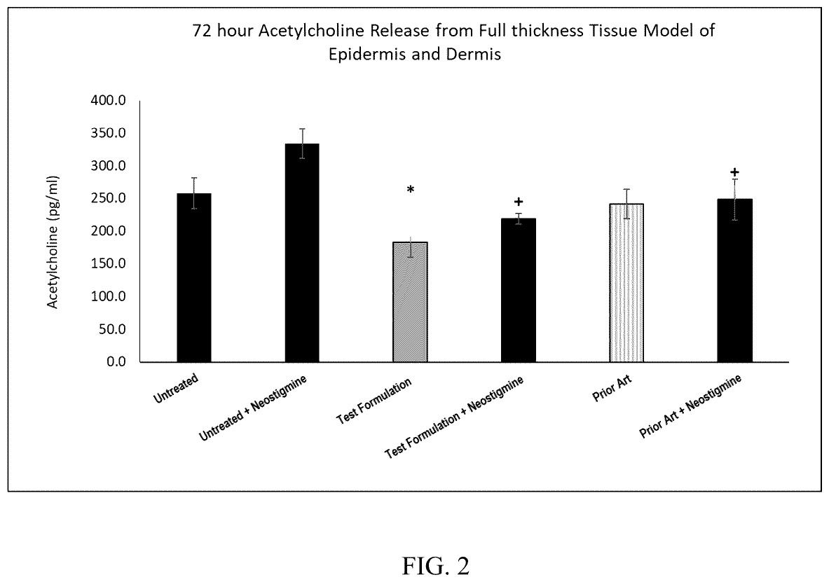

FIG. 2 is a chart comparing results of the test formulation for multi-modal reduction of ACh concentration according to an embodiment of this disclosure with results derived from a prior art formulation providing only two mechanisms of action. The results show the ACh concentration in picograms per milliliter at 72 hours. While the results in the chart in FIG. 2 depict higher concentrations of ACh at 72 hours as compared to the results in the chart in FIG. 1, the same general trend can be observed: the tissue model treated with the test formulation resulted in a decreased concentration of ACh as compared with the untreated tissue model, and presence of neostigmine resulted in relatively higher concentrations of ACh in the untreated tissue model and the tissue model treated with the test formulation, i.e., Test formulation+Neostigmine. Further, the tissue model exposed to neostigmine and the test formulation still resulted in a lower concentration of ACh than the untreated tissue model.

Two additional tissue models were prepared to test a prior art skincare formulation that only included two neuromodulating peptides offering two mechanisms of action, at the motor neurons and the muscle cells. As can be seen, the tissue model treated with the prior art skincare formulation resulted in a higher concentration of ACh in the tissue culture media than the tissue models treated with the test formulation or the tissue model treated with the test formulation in the presence of Neostigmine. Thus, the results depicted in FIG. 2 indicate that the test formulation that includes an inhibitory neurotransmitter and a plurality of neuromodulating peptides that provides multiple mechanisms targeting three different locations provides an improvement in reduction of ACh concentration and activity over prior formulations that include fewer neuromodulating peptides offering fewer mechanisms of action targeting only two different locations.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A method for reducing facial expression lines by attenuating muscle contraction via multimodal reduction of acetylcholine (ACh) concentration and activity at a plurality of different locations, the method comprising:
reducing neuronal excitability;
increasing competition with SNAP-25 in a SNARE complex of a motor neuron, at a presynaptic terminal;
destabilizing the SNARE complex of the motor neuron, at the presynaptic terminal;
inhibiting fusion of ACh vesicles within a membrane of the motor neuron at the presynaptic terminal;
inhibiting contraction of muscles via inhibiting ion channel communication in a muscle cell, at the post-synaptic terminal; and
blocking reception of ACh at a postsynaptic membrane.

2. The method of claim 1, wherein reducing neuronal excitability comprises binding an inhibitory neurotransmitter to an inhibitory neurotransmitter receptor on an epidermal layer.

3. The method of claim 2, wherein the inhibitory neurotransmitter is gamma aminobutyric acid.

4. The method of claim 1, wherein increasing competition with SNAP-25 in the SNARE complex of the motor neuron comprises mimicking the N-terminal end of SNAP-25.

5. The method of claim 4, wherein the N-terminal end of SNAP-25 is mimicked by acetyl octapeptide-3.

6. The method of claim 1, wherein destabilizing the SNARE complex of the motor neuroncomprises mimicking the N-terminal end of SNAP-25.

7. The method of claim 6, wherein the N-terminal end of SNAP-25 is mimicked by acetyl hexapeptide-8.

8. The method of claim 1, wherein inhibiting fusion of ACh vesicles within the membrane of the motor neuron comprises inactivating SNAP-25 from the SNARE complex.

9. The method of claim 8, wherein the fusion of the ACh vesicles is inhibited by palmitoyl hexapeptide-19.

10. The method of claim 1, wherein inhibiting contraction of muscles via inhibiting ion channel communication in the muscle cell comprises blocking muscular nicotinic ACh membrane receptors to inhibit sodium ion transfer.

11. The method of claim 10, wherein blocking muscular nicotinic ACh membrane receptors comprises mimicking the essential amino acid sequence of a Waglerin-1 functionality.

12. The method of claim 11, wherein the essential amino acid sequence of the Waglerin-1 functionality is mimicked by Dipeptide Diaminobutyroyl Benzylamide Diacetate.

13. The method of claim 1, wherein blocking reception of ACh at a postsynaptic membrane comprises inhibiting formation of the SNARE complex by preventing Syntaxin from binding to Munc-18 for formation of the SNARE complex.

14. The method of claim 13, wherein binding of the Syntaxin to the Munc-18 is prevented by acetyl hexapeptide-1.

15. The method of claim 1, wherein blocking reception of ACh at a postsynaptic membrane further comprises blocking the reception of ACh at a postsynaptic membrane to reduce clustering of ACh receptors and destabilizing the ACh receptors to inhibit calcium ion transfer.

16. The method of claim 15, wherein the reception of the ACh at the postsynaptic membrane is blocked by acetyl hexapeptide-1.
